(19) United States
(12) Patent Application Publication
(10) Pub. No.: US 2009/0162927 A1
(43) Pub. Date: Jun. 25, 2009

(54) NANOTUBES AND NANOWIRES BASED ELECTRONIC DEVICES AND METHOD OF FABRICATION THEREOF

(75) Inventors: Ron Naaman, Rehovot (IL); Miron Hazani, Haifa (IL); Dmitry Shvarts, Kiryat Ono (IL); Dana Peled, Rehovot (IL); Victor Sidoriv, Haifa (IL)

Correspondence Address: BROWDY AND NEIMARK, P.L.L.C.;624 NINTH STREET, NW, SUITE 300, WASHINGTON, DC, 20001-5303, US

(73) Assignee: Yeda Research and Development Company Ltd., Rehovot (IL)

(21) Appl. No.: 11/443,441

(22) Filed: May 31, 2006

(51) Int. Cl. C12M 1/00; G01N 27/00

(52) U.S. Cl. 435/287.1; 422/82.01

(57) ABSTRACT

An electronic device is presented comprising at least one electrically conductive element coupled to an elongated carbon or inorganic semiconductor based nanostructure, by a biological binder.

### FIELD OF THE INVENTION

This invention is generally in the field of biomolecular electronics, and relates to a nanotubes or nanowires based electronic device and method of its fabrication.

### LIST OF REFERENCES

The following references are considered to be pertinent for the purpose of understanding the background of the present invention:
[1] S. G. Rao, L. Huang, W. Setyawan, S. Hong, Nature 425 (2003) 36;
[2] B. Kim, W. M. Sigmund, Langmuir 19 (2003) 4848;
[3] G. Xia, H. Tengjiao, L. Luqi, G. Zhixin, Chem. Phys. Lett. 370 (2003) 661;
[4] Z. Liu, Z. Shen, J. Zhu, S. Hou, L. Ying, Z. Shi, Z. Gu, Langmuir 16 (2000) 3569;
[5] H. Xin, A. T. Wooley, J. Am. Chem. Soc. 125 (2003) 8710;
[6] K. Keren, R. S. Berman, E. Buchstab, U. Sivan, E. Braun, Science 302 (2003) 1380;
[7] M. Hazani, R. Naaman, F. Hennrich, M. M. Kappes, Nano Lett. 3 (2003) 153;
[8] M. S. Strano, C. A. Dyke, M. L. Usrey, P. W. Barone, M. J. Allen, H. Shan, C. Kittrell, R. H. Hauge, J. M. Tour, R. E. Smalley, Science 301 (2003) 1519;
[9] M. Zheng, A. Jagota, M. S. Strano, A. P. Santos, P. Barone, S. G. Chou, B. A. Diner, M. S. Dresselhaus, R. S. Mclean, G. B. Onoa, G. G. Samsonidze, E. D. Semke, M. Usrey, D. J. Walls, Science 302 (2003) 1545;
[10] J. Appenzeller, J. Koch, V. Derycke, R. Martel, S. Wind, Ph. Avouris, Phys. Rev. Lett. 89 (2002) 126 801;
[11] S. Heinze, J. Tersoff, R. Martell, V. Derycke, J. Appenzeller, Ph. Avouris, Phys. Rev. Lett. 89 (2002) 106 801;
[12] L. Marty, V. Bouchiat, C. Naud, M. Chaumont, T. Fournier, A. M. Bonnoy, Nano Lett. 3 (2003) 1115;
[13] S. J. Wind, J. Appenzeller, Ph. Avouris, Phys. Rev. Lett. 91 (2003) 058301;
[14] S. Tans, A. R. Vershueren, and C. Dekker, Nature (London) 393, 49 (1998);
[15] R. Martel, T. Schmidt, H. R. Shea, T. Hertel, and P. H. Avouris, Appl. Phys. Lett. 73, 2447 (1998);
[16] S. J. Wind, J. Appenzeller, R. Martel, V. Derycke, and Ph. Avouris, Appl. Phys. Lett. 80, 3817 (2002);
[17] Ph. Avouris, R. Martel, V. Derycke, and J. Appenzeller, Physica B 323, 6 (2002);
[18] A. Javey, H. Kim, M. Brink, Q. Wang, A. Ural, J. Guo, P. McIntyre, P. McEuen, M. Lundstrom, and H. Dai, Nat. Mater. 1, 241 (2002);
[19] J. Appenzeller, J. Knoch, R. Martel, V. Derycke, S. Wind, and Ph. Avouris, IEEE Trans. Nanotechnol. 1, 184 (2002);
[20] V. Derycke, R. Martel, J. Appenzeller, and Ph. Avouris, Appl. Phys. Lett. 80, 2773 (2002);
[21] L. Yung-kuo, Problems and Solutions on Electromagnetism (World Scientific, Singapore, 1993);
[22] H.-M. Mühlhoff, and D. V. McCaughan, in Device Physics, edited by C. Hilsum (Elsevier, Amsterdam, 1993).
[23] V. Derycke, R. Martel, M. Radosavljevic, F. M. Ross, P. Avouris, Nano Letters, 2002, 2, 1043.
[24] S. Orlanducci, V. Sessa, M. L. Terranova, M. Rossi, D. Manno, Chem. Phys. Lett., 2003, 367, 109.
[25] S. M. Huang, X. Y. Cai, J. Liu, J. Am. Chem. Soc. 2003, 125, 5636.
[26] L. Ki-Hong, C. Jeong-Min, W. Sigmund, App. Phys. Lett., 2003, 82, 448.
[27] S. J. Wind, R. Martel, Ph. Avouris, J. Vac. Sci. & Tech. B, 2002, 20, 2745.
[28] P. Mauron, C. Emmenegger, A. Zuttel, C. Nutzenadel, P. Sudan, L. Schlapbach, Carbon, 2002, 40, 1339.
[29] N. Bendiab, R. Almairac, J. L. Sauvajol, S. Rols, E. Elkaim, J. Appl. Phys. 2003, 93, 1769.
[30] L. Jin, C. Bower, O. Zhou, Appl. Phys. Lett. 1998, 73, 1197.
[31] Y. T. Jang, J. H. Ahn, B. K. Ju, Y. H. Lee, Solid state Comm. 2003, 126, 305.
[32] T. Ono, E. Oesterschulze, G. Georgiev, A. Georgieva, R. Kassing, Nanotechnology, 2003, 14, 37.
[33] Y. Zhang, A. Chang, J. Cao, Q. Wang, W. Kim, Y. Li, N. Morris, E. Yenilmez, J. Kong, H. Dai. App. Phys. Letts, 2001, 79, 3155.
[34] K. Yamamoto, S. Akita, Y. Nakayama, J. Phys. D-Applied Physics, 1998, 31, L34.
[35] R. Krupke, F. Hennrich, H. Weber, D. Beckmann, O. Hampe, S. Malik, M. Kappes, H. von Löhneysen, Appl. Phys. A, 2003, 76, 397.
[36] J. E. Fischer, W. Zhou, J. Vavro, M. C. Llaguno, C. Guthy, R. Haggenmueller, M. J. Casavant, D. E. Walters, R. E. Smalley, J. App. Phys., 2003, 93, 2157;
[37] B. W. Smith, Z. Benes, D. E. Luzzi, J. E. Fischer, D. A. Walters, M. J. Casavant, J. Schmidt, R. E. Smalley, App. Phys. Lett, 2000, 77, 663.
[38] M. Fujiwara, E. Oki, M. Hamada, Y. Tanimoto, I. Mukouda, Y. Shimomura, J. Phys. Chem. A, 2001, 105, 4383.
[39] X. Gao, T. o Hu, L. Liu, Z. Guo, Chem. Phys. Lett. 2003, 370, 661.
[40] R. Krupke, F. Hennrich, H. von Löhneysen, M. M. Kappes, Science, 2003, 301, 344.
[41] M. Stadermann, S. J. Papadakis, M. R. Falvo, J. Novak, E. Snow, Q. Fu, J. Liu, Y. Fridman, J. J. Boland, R. Superfine, S. Washburn, Physical Review, B 2004, 69, 201402.
[42] T. Durkop, B. M. Kim, and M. S. Fuhrer, J. Phys.: Cond. Matt. 2004, 16, R553.
[43] M. S. Fuhrer, B. M. Kim, T. Durkop, and T. Brintlinger, Nano. Lett. 2002, 2, 755.
[44] C. H. Lee, K. T. Kang, K. S. Park, M. S. Kim, H. S. Kim, H. G. Kim, J. E; Fischer, and A. T. Johnson, Jpn. JAppl. Phys. 2003, 42, 5392.
[45] W. Kim, A. Javey, O. Vermesh, Q. Wang, Y. Li, H. Dai, Nano. Lett. 2003, 3, 193.
[46] T. Durkop, S. A. Getty, E. Cobas, and M. S. Fuhrer, Nano. Lett. 2004, 4, 35.
[47] P. G. Collins, M. Hersam, M. Arnold, R. Martel, and Ph. Avouris, Phys. Rev. Lett. 2001, 86, 3128.

### BACKGROUND OF THE INVENTION

Carbon and/or inorganic semiconductor nanotubes have been proposed to be used as building blocks for nanofabrication of electronic devices and sensors. Successful integration of such nanotubes in such devices requires controlled deposition at well defined locations and appropriate electrical contacts to metal leads.

Various techniques aimed at achieving controlled deposition of carbon nanotubes have recently been developed, including directional growth of tubes, alignment by mechanical forces, alignment by an electric field, alignment by a magnetic field, the use of a patterned assembly, and the use of a self-assembly [1, 2, 3, 4]. Controlled self-assembly of carbon nanotubes can be achieved by interphasing them with biological molecules [5].

The fabrication of a DNA-templated carbon nanotube field effect transistor have recently been reported [6]. According to this technique, a long DNA molecule featuring RecA proteins is used as a scaffold onto which streptavidin—functionalized single-walled carbon nanotubes (SWNTs) are assembled, utilizing anti-RecA primary antibodies and biotinylated secondary antibodies. Electrical contact to the tubes is achieved by metallizing the scaffold DNA molecule.

Based on the discovery of carbon nanotubes and especially the development of carbon nanotubes based field-effect transistors (CNTFET) [14, 15], recent advances in the CNTFET technology allow the production of carbon nanotubes based devices with performances comparable to those of state-of-the-art silicon MOSFETs [16-18].

Successful incorporation of carbon nanotubes into integrated circuits requires techniques for controlled deposition of millions of carbon nanotubes in predefined locations. However, most of the carbon nanotubes based devices described in the literature are based on single nanotube manipulation methods, which are incompatible with large-scale production. Self-assembly, based on molecular recognition, was proposed as a promising method for batch-like positioning of carbon nanotubes [1, 5, 6].

US patent publication No. 2002/0027124 discloses a method of assembling a nanometer-scale construct. The method comprises: (a) providing a nanometer-scale object such as an active electron device; (b) attaching a first bio-link to said nanometer-scale object to form a functionalized nanometer-scale object; (c) providing a substrate; (d) attaching a second bio-link to said substrate to form a functionalized substrate, wherein said second bio-link is a complement to said first bio-link in that said second bio-link selectively binds with said first bio-link; and (e) bringing said functionalized nanometer-scale object within close enough proximity of said functionalized substrate that said second bio-link selectively binds with said first bio-link, and thereby forms an assembled nanometer-scale construct.

US patent publication No. 2004/0046002 discloses a self assembled nano-devices using DNA. According to this technique, an article of manufacture includes an organic structure and inorganic atoms bonded to specific locations on the organic structure.

US patent publication No. 2004/0115696 discloses methodologies and techniques that utilize programmable functionalized self-assembling nucleic acids, nucleic acid modified structures, and other selective affinity or binding moieties as building blocks for creating molecular electronic and photonic mechanisms; organizing, assembling, and interconnecting nanostructures, submicron- and micron-sized components onto silicon or other materials; organizing, assembling, and interconnecting nanostructures, submicron- and micron-sized components within perimeters of microelectronic or optoelectronic components/devices; and creating and manufacturing photonic and electronic structures, devices, and systems. This technique provides for forming a multiple identity substrate material. According to this method, a first affinity sequence is provided at multiple locations on a support. A functionalized second affinity sequence is provided, which reacts with the first affinity sequence, and has an unhybridized overhang sequence. First affinity sequences and second affinity sequences are selectively cross-linked.

### SUMMARY OF THE INVENTION

There is a need in the art to facilitate production of self-assembled nanostructures based devices with high efficiency, especially in a manner facilitating mass production of such devices. This is associated with the fact that the known techniques of the kind specified, despite their ability to fabricate single-walls carbon nanotube based devices, involve multiple production steps resulting in low yield.

The present invention solves the above problem by providing a novel method for producing self-assembled nanostructures based devices. The term “nanostructure” used herein refers to a carbon and/or inorganic semiconductor elongated nanostructure, such as nanotube or nanowire or nanotubes and/or nanowires' bundle and/or network of such nanowires or nanotubes or of their bundles. Such a nanostructure may include a biological binder coupling the nanowires or nanotubes into the network.

The invention takes advantage of a technique of covalently modifying single-walled carbon nanotubes with DNA molecules [7], and utilizes a modification of this technique to adsorb nanotubes via biological moieties to an electrically conductive element (e.g., gold electrode) taking advantage of a specific binding ability of biological moieties (such as the specific binding between complementary DNA strand and thus causing natural hybridization between them).

According to the present invention, the biological moiety may be any biological moiety that is capable of interacting specifically, directly or indirectly, with a recognizable moiety, which may be a biological moiety or an inorganic substance, to form stable biological binding. The biological moiety would be bound to the nanostructure, and the recognizable moiety would be bound to the electric conductive layer, or vice versa.

The technique of the invention presents a straightforward, two step method for self-assembling carbon and/or inorganic semiconductor elongated nanostructure between electrodes. The technique utilizes a biological binder between an electrically conductive element (metal contact) and an elongated nanostructure, namely a specific binding of synthetic or natural biological moieties (such as hybridization between short complementary sequences) located on the metal contacts and the nanostructure.

This technique enables simple production of hundreds of devices with high yields. The electrical characteristics depend strongly on the existence of the chemical binding group (namely, a contact with the surface other than that through the binding molecules does not produce an electrical contact; or a direct electrical contact is established by removal of the binding molecules by the way of heating, as the case may be), and are controlled by the transport through these groups. The electric current measured is larger by two orders of magnitude than the values reported for direct metal-nanotube contacts.

Moreover, the present invention provides a novel technique for incorporation of elongated nanostructures into integrated circuits, enabling a high yield production of such nanostructures based field effect transistors.

There is thus provided according to one aspect of the invention, an electronic device comprising at least one electrically conductive element coupled to a carbon or inorganic semiconductor based elongated nanostructure, by a biological binder.

The biological binder may be used for coupling between the electrically conductive element and the nanostructure, and then removed (by thermal treatment) to form a direct contact between them. Likewise, an electronic device can comprise an electrically conductive element having at least one direct contact with a carbon or inorganic semiconductor elongated nanostructure.

The device may include the first and second spaced-apart electrically conductive elements connected to each other via the nanostructure. Each end of the nanostructure is coupled to the respective one of the first and second elements by the first and second binding moieties forming the biological binder. At least one of the binding moieties is a biological moiety. The biological binder may be formed as a result of natural hybridization of free ends of two complementary single stranded nucleic acids sequences, opposite ends of which being adsorbed to, respectively, the electrically conductive elements and the respective ends of the elongated nanostructure.

The device may be configured as a transistor device (FET) formed by source and drain electrodes connected to each other via the nanostructure (as described above), which defines a transistor channel, and a gate electrode on dielectric. The electrodes' arrangement (source, drain and gate) may be formed in a layer structure including a semiconductor substrate carrying a continuous layer of the dielectric, and the source and drain electrodes arranged in the spaced-apart relationship on top of this dielectric layer, such that the semiconductor substrate serves as the gate electrode on the dielectric. Alternatively, the layer structure may include a semiconductor substrate carrying two spaced-apart regions of the dielectric spaced by a region of the substrate, and the source and drain electrodes on top of the dielectric regions, respectively. In this case, a native oxide of the surface region of the substrate within the space between the dielectric regions serves as the dielectric for the gate electrode defined by the inner material of the semiconductor substrate.

According to another broad aspect of the invention, there is provided an electronic device comprising at least first and second spaced-apart electrodes connected to each other via a carbon or inorganic semiconductor based elongated nanostructure, wherein each end of the nanostructure is coupled to the respective one of the first and second electrodes by means of a biological binder, serving as a chemical linker, the biological binder being formed by a first moiety adsorbed onto the respective electrode and a second moiety recognizable by said first moiety and adsorbed onto the respective end of the nanostructure, at least one of the first and second moieties being a biological moiety.

According to yet another broad aspect of the invention, there is provided a field effect transistor device comprising spaced apart source and drain electrodes, a gate electrode on a dielectric, and an elongated nanostructure interconnected between the source and drain electrodes thereby forming a transistor channel, opposite ends of said nanostructure being connected to the source and drain electrodes, respectively, via two biological binders, the biological binder being formed by a first moiety adsorbed onto the respective electrode and a second moiety recognizable by said first moiety and adsorbed onto the respective end of the nanostructure, at least one of the first and second moieties being a biological moiety.

According to yet another aspect of the invention, there is provided a method for manufacturing an electronic device formed by at least one electrically conductive element coupled to a carbon or inorganic semiconductor based elongated nanostructure by a biological binder, the method consisting of the following steps:

(i) adsorbing onto a surface of at least one electrically conductive element a self-assembled monolayer of a first binding moiety that is to form at least one biological binder;

(ii) providing the elongated nanostructure, modified at its opposite ends with one or more second binding moiety of a kind recognizable by the first moiety, where at least one of the first and second binding moieties types is a biological moiety type, and allowing coupling between the recognizable first and second binding moieties thereby forming said at least one biological binder acting as a chemical linker between the end of the nanostructure and the electrically conductive element.

In yet another aspect of the invention, there is provided a method for manufacturing an electronic device formed by at least one electrically conductive element coupled to a carbon or inorganic semiconductor based elongated nanostructure, the method consisting of the following steps:

(i) adsorbing onto a surface of at least one electrically conductive element a self-assembled monolayer of a first binding moiety that is to form at least one biological binder;

(ii) providing the elongated nanostructure, modified at its opposite ends with one or more second binding moiety of a kind recognizable by the first moiety, where at least one of the first and second binding moieties types is a biological moiety type, and allowing coupling between the recognizable first and second binding moieties thereby forming said at least one biological binder acting as a chemical linker between the end of the nanostructure and the electrically conductive element.

(iii) applying a thermal treatment to destruct the biological moieties or the biological binder thereby forming a direct contact between the at least one electrically conductive element and the end of the elongated nanostructure.

The invention also provides a method of manufacturing an array of the above-described electronic devices, enabling the mass production of such devices. The method comprises:

providing an array of the electrically conductive elements on a substrate, each element being separately addressed by a voltage supply thereto;

selectively adsorbing first biological moieties of different kinds onto surfaces of the different electrically conductive elements, by maintaining all the electrically conductive elements at a certain voltage, except for at least one selected element onto which the moiety is to be adsorbed, thus allowing adsorption of said moiety solely to said at least one selected element;

providing an array of the elongated nanostructures modified with second moieties recognizable by the first moieties, and allowing coupling between the recognizable first and second moieties, thereby forming a plurality of the biological binders between the electrically conductive elements and the elongated nanostructures defining the array of the electronic devices.

Optionally, the method of manufacturing an array of the electronic devices can include applying a thermal treatment to destruct the formed biological moieties or the biological binder, thereby forming at least one direct contact between at least one electrically conductive element and an end of an elongated nanostructure.

### BRIEF DESCRIPTION OF THE DRAWINGS

In order to understand the invention and to see how it may be carried out in practice, embodiments of the invention will now be described, by way of non-limiting examples only, with reference to the accompanying drawings, in which:

FIGS. 1A and 1B exemplify an electronic device of the present invention and a method of its fabrication;

FIGS. 2A and 2B show the SEM images of, respectively, two electrode pairs connected by elongated nanostructures (nanotubes); where in FIG. 2A the two sides of the nanotube are bound chemically to the electrodes, and in FIG. 2B one side (on the right) is not chemically bound;

FIG. 2C shows the I-V characteristics corresponding to the images of FIGS. 2A and 2B; illustrating that the device of FIG. 2A has a characteristic I-V curve, while in the device of FIG. 2B no electric current is measured;

FIGS. 3A and 3B show I-V measurements of different electrode pairs connected using nanotubes modified with oligonucleotides complementary to the sequences present on the electrodes (FIG. 3A) and non-complementary oligonucleotides (FIG. 3B), where dotted traces correspond to cases where no SEM indication for electrical contact appears, as in FIG. 2B;

FIGS. 4A and 4B schematically illustrate two types of a transistor device of the present invention;

FIG. 5 illustrates I_{sd}−V_{sd} curves for different gate voltages of the transistor device with a 100 nm gate dielectric layer (device of FIG. 4A); the insert to FIG. 5 shows the conductance at zero V_{sd} for devices of FIGS. 4A and 4B, where graph R₁ correspond to the 100-nm-thick SiO₂ gate layer device of FIG. 4A, and graph R₂ corresponds to the native oxide device of FIG. 4B;

FIGS. 6A and 6B show I_{sd}−V_{g} curves for different gate voltages V_{g}, for the devices of FIGS. 4A and 4B, respectively;

FIG. 7 illustrates an inverse subthreshold slope, S, as a function of source-drain voltage V_{sd} for the devices of FIGS. 4A and 4B;

FIGS. 8A and 8B exemplify a technique of the present invention for selectively coupling different nucleic acid sequences to different electrodes; and

FIGS. 9A to 9C exemplify a technique of the present invention for coupling nucleic acid sequences to the ends of nanotubes.

FIGS. 10A and 10C show the I_{sd}−V_{sd} curves for two devices each consisting of an electrode pair connected by an elongated nanostructure in the form of a nanotube network providing multiple conducting channels between the electrode pair; and FIGS. 10B and 10D show the SEM images of these two devices.

FIGS. 11A and 11B show, respectively, the I_{sd}−V_{sd} curve for another device consisting of an electrode pair connected by an elongated nanostructure and the SEM image of this device.

FIG. 12 demonstrates two I_{sd}−V_{sd} curves, one obtained before and one after a mild thermal treatment, for a device which network contains multiple conducting channels for connecting two electrodes (for a device similar to the devices shown in FIGS. 10A-10D).

FIGS. 13A to 13D present scanning electron microscopy (SEM) images of two devices conducting through SWNT networks (FIGS. 13A and 13C), and illustrate an effect of high currents on these devices (FIGS. 13B and 13D).

FIG. 14 demonstrates hysteresis curves (I_{sd} vs V_{g}) measured by the inventors for sweeps of the gate voltage in different ranges. In the insert to FIG. 14 the dependence of the threshold voltage on the starting voltage is shown.

FIG. 15A presents the hysteresis loops measured with different delays between the voltage setting and the current measurement;

FIG. 15B shows several write-read cycles of the invented CNTFETs while switching the gate voltage between +20 V, 0 V and −20 V;

FIG. 15C presents the variation of the device current on long-range scale while switching the gate voltage between +20 V and −20 V.

### DETAILED DESCRIPTION OF THE INVENTION

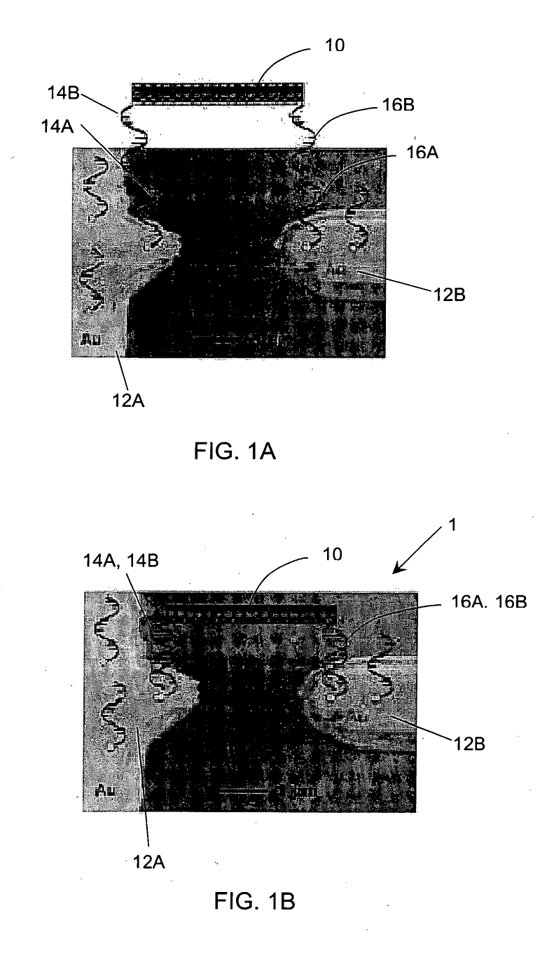

Referring to FIGS. 1A and 1B, there is exemplified a nanotube based (carbon and/or inorganic semiconductor nanotube) electronic device of the present invention and a method of its fabrication. In the present example, a carbon nanotube based device is considered. It should be noted that the term “nanotube” used in the description below refers also to a nanowire and nanotubes' and/or nanowires' bundle.

As shown in FIG. 1B, an electronic device 1 is formed by a carbon nanotube 10 interconnected between two electrodes 12A and 12B (gold electrodes in the present example) via a biological binder formed by nucleic acid bindings 14A-14B and 16A-16B, being binding biological moieties that serve as chemical linkers.

As indicated above, the biological moiety according to the present invention may be any biological moiety of the kind that is known to specifically interact with another recognizable moiety, which may or may not be a biological moiety. In the context of this invention, the biological moiety may be for example a peptide based molecule (peptide, polypeptide or protein, glycoprotein, etc.) or a nucleic acid based moiety (e.g. an oligonucleotide or polynucleotide, preferably having a sequence of between 5 to 30 bases) or any modification thereof. The recognizable moiety may be a second biological moiety or an inorganic moiety capable of specifically binding to the biological moiety.

Accordingly, the interaction between the first biological moiety and the second recognizable moiety may include protein/protein, nucleic acid/protein or nucleic acid/nucleic acid interaction. Some non-limiting examples of such binding moieties include enzyme/substrate, antigen/antibody, ligand/receptor, nucleic acid sequence/nucleic acid sequence, nucleic acid sequence/nucleic acid biding proteins, sugar/lectin, enzyme/inhibitor, enzyme/co-factor etc.

The term “nucleic acid binding” means an interaction between complementary strands of nucleic acids sequences. Each of the bound nucleic acid sequences may be a sequence composed of DNA nucleotides, RNA nucleotides or a combination of both types, and may include natural nucleotides, chemically modified nucleotides and synthetic nucleotides. Accordingly, binding pairs would include DNA-DNA interactions, DNA-RNA interactions, RNA-RNA interactions, etc. In fact, nucleic acid binding in the present invention may occur between two or more nucleic acid sequences. Whilst the following examples depict use of nucleic acid sequences as linking biological moieties, the present invention is not limited thereto, as a person skilled in the art of the invention would easily modify the procedure to use other biological moieties (such as proteins).

As indicated above, such a nucleic acid binding is the interaction between complementary strands of nucleic acids sequences; each of the bound nucleic acid sequences may be a sequence composed of DNA nucleotides, RNA nucleotides or a combination of both types and may includes natural nucleotides, chemically modified nucleotides and synthetic nucleotides.

As mentioned above, the interaction between the first biological moiety and the second recognizable moiety may also be indirect, involving two or more specific biological interactions. For example, the nanotube bound biological moiety and the electric conductive layer bound recognizable moiety may be two oligonucleotides that are not complementary to each other. Nevertheless, they may both bind specifically and indirectly via a third oligonucleotide or protein that has one portion that binds to the first oligonucleotide and one portion that binds to the second oligonucleotide.

Finally, a person skilled in the art of the invention would be able to provide the necessary conditions (pH, temperature, required chemical agents, etc.) that would be necessary to enable the binding to take place at sufficient specificity to reduce (preferably minimize) non-specific interactions.

Device 1 is manufactured by a two-step method consisting of the following:

In the first step, single stranded DNA oligonucleotides (ssDNA) 14A and 16A (constituting biological moieties), which in this example have the same nucleotide sequence, are adsorbed onto gold contacts 12A and 12B, respectively. In this example, the ssDNA is thiolated, thus enabling a reaction between the thiol terminus and the gold contact, which causes the binding of the ssDNA to the gold contact, yielding a self-assembled monolayer of ssDNA.

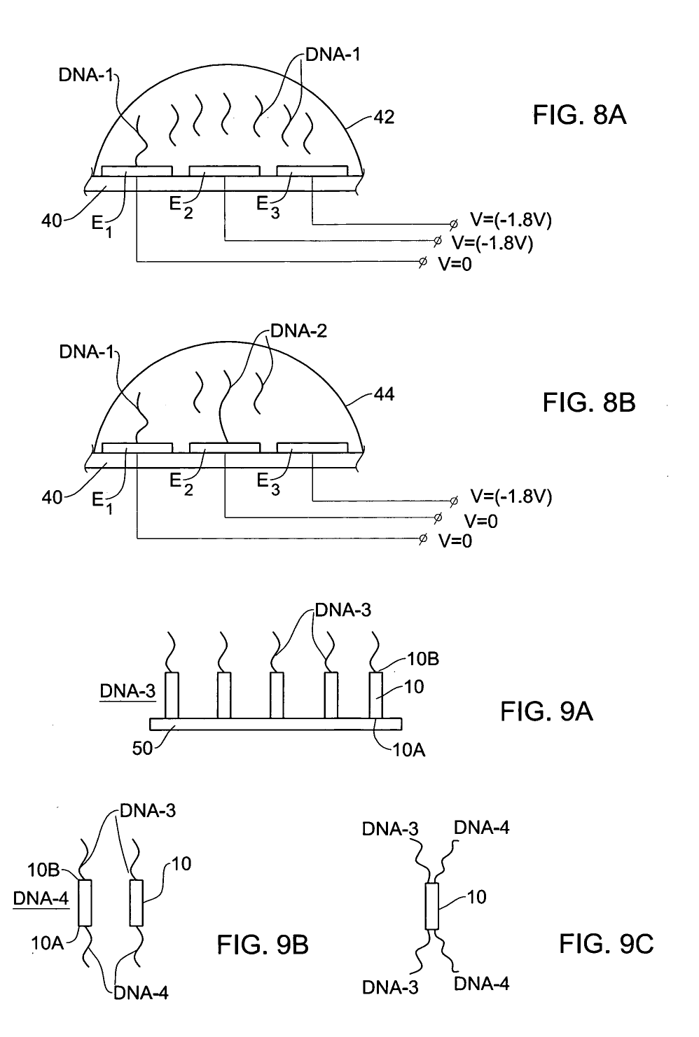

The inventors have developed a novel technique for using different nucleic acid sequences at each end of the nanotubes and electrodes (which is particularly useful in cases where the electrodes are very close to each other), thereby facilitating mass production of electronic devices. This will be described further below with reference to FIGS. 8A-8C and 9A-9C.

More specifically, the formation of the self-assembled monolayer of ssDNA was as follows: Samples bearing gold electrodes 12A and 12B were cleaned by successive sonication in acetone, methanol, and isopropanol (5 min each) followed by extensive washing in de-ionized water and drying. The samples were then placed in a UV-ozonator for 20 min, followed by immersion in ethanol for another 20 min. Then, the samples were washed with ethanol, dried and immediately covered with a few drops from a solution of 1 μM 3-thiolated oligonucleotides in 0.4M phosphate buffer, pH 7. After over-night incubation, the samples were washed by successive immersions in 0.4M phosphate, buffer pH7.

In the next step, nanotubes 10 modified with ssDNA oligonucleotides 14B and 16B (constituting the second recognizable moieties, biological moieties in the present example) that are complementary to sequences 14A and 16A, respectively, are allowed to hybridize with the ssDNA 14A and 14B located on the gold contacts.

More specifically, the nanotubes were chemically activated by oxidation 10 and then bound to 3-amino modified oligonucleotides with modifications to the procedure described previously [7]. According to the example of the invention, oxidized nanotubes 10 suspended in 0.1M phosphate buffer, pH 7, were reacted with 5 μm 3 amino terminated oligonucleotides in the presence of 100 mM 1-ethyl-3-(3-dimethylamino-propyl) carbodiimide and 10 mM N-hydroxysuccinimide, for 12 hours at room temperature. The reaction mixture was filtered over a 0.2 μm polycarbonate membrane, washed with 100 ml of de-ionized water and resuspended in H₂O. Deposition onto electrodes 12A and 12B was achieved by immersing the samples in an aqueous suspension of the ssDNA modified nanotubes 10 containing 0.1M phosphate buffer, pH 7, and 0.5M NaCl, at room temperature for 12 hours. The samples were then extensively washed by successive immersions in fresh hybridization buffer followed by washing with a solution of 0.1M N-2-hydroxyethylpiperazine-N-2-ethansulphonic acid (Hepes), pH 7, 0.5M NaCl. Finally, the samples were washed briefly with H₂O and dried by nitrogen stream.

Following are the experimental results of the technique of the present invention:

Arrays of hundreds of electrodes bearing ssDNA were exposed to nanotubes modified with ssDNA sequences complementary or non-complementary to the DNA sequences that were bound to the electrodes, as described, and characterized by scanning electron microscopy (SEM) and room temperature current-voltage measurements.

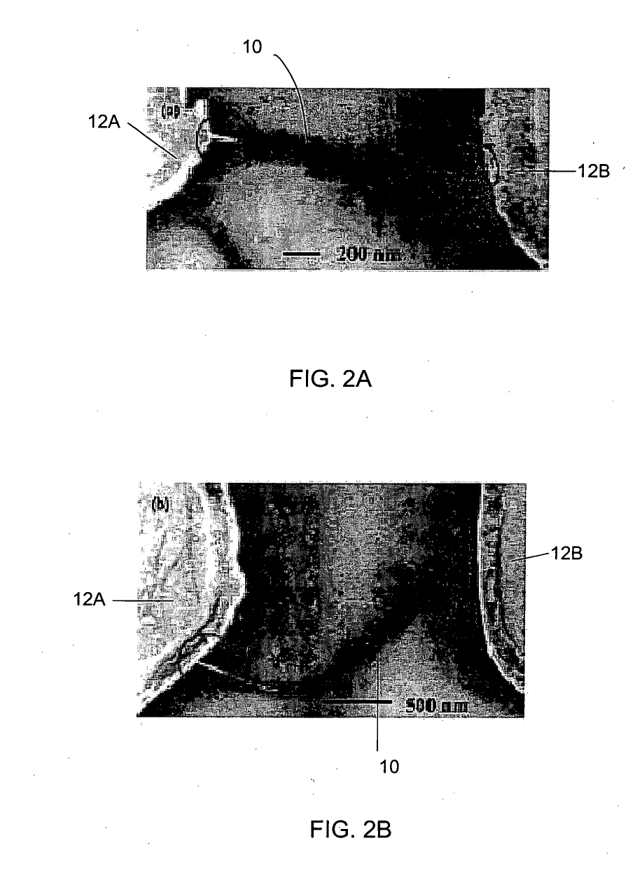

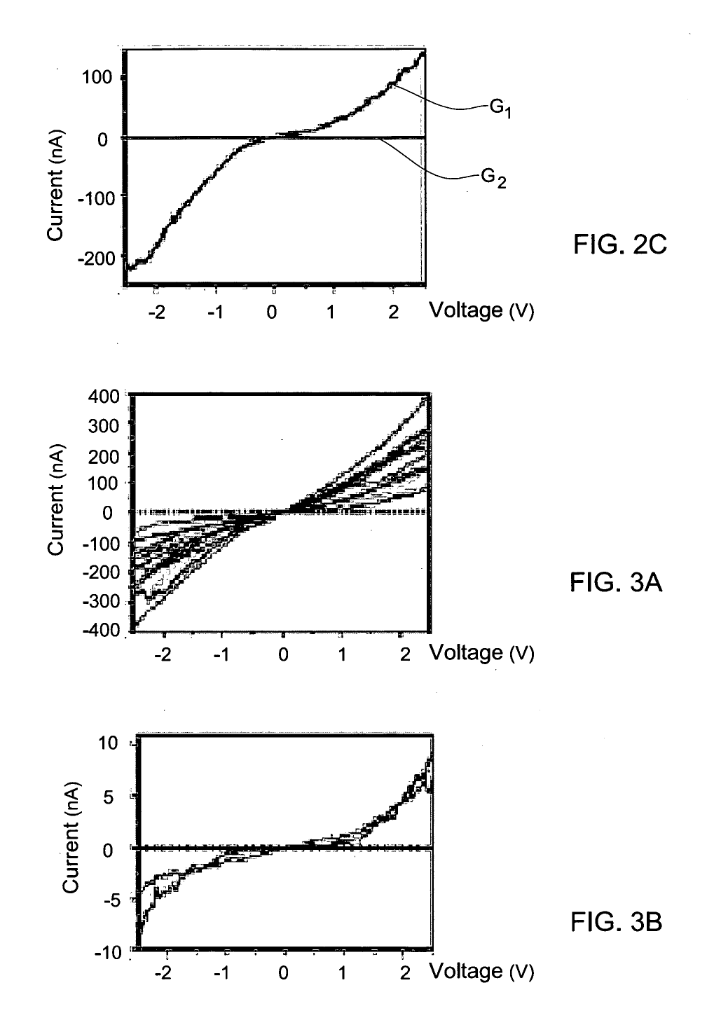

FIGS. 2A and 2B illustrate SEM images of two devices of the present invention. It should be understood that the term “device” used herein refers to a pair of electrodes connected by a nanotube or a nanotubes bundle via nucleic acid sequences. Thus, each illustrated device is formed by a pair of electrodes 12A and 12B connected by a nanotube 10 via double stranded DNA (not shown). The SEM images show a variation in the appearance of nanotubes 10 with respect to a distance from the electrodes 12A and 12B. FIG. 2C shows two graphs G₁ and G₂ presenting the current versus voltage tracings corresponding to the images of FIGS. 2A and 2B, respectively.

The circled areas in FIGS. 2A-2B show transitions from thin features, observed close to the electrode, to thicker features observed further away from the electrodes 12A and 12B. The thickness was found to be dependent on an acceleration voltage used in the SEM (scanning electron microscope). In FIG. 2A, nanotube 10 appears thin and bright close to both electrodes 12A and 12B, whereas in FIG. 2B, nanotube 10 has such appearance only close to electrode 12A. This change in appearance is a result of charging of the DNA present on nanotube 10, and depends on the electron beam energy. The fact that the charge increases with the distance from electrodes 12A and 12B and dissipates close to them is associated with the nature of the contact between the electrodes and the nanotubes. The distance dependence indicates the non-ohmic nature of the coupling between the nanotubes 10 and the electrically conductive layers 12A and 12B.

The inventors have found that an electric current between electrodes 12A and 12B can be measured only when an indication for electrical contact in the SEM image appears, namely the appearance of uncharged regions on nanotubes 10 in proximity to both electrodes 12A and 12B. Measurements in the range of ±2V could be repeated for hundreds of times without destroying the device.

To assess non-specific interactions, gold electrodes covered with a ssDNA monolayer were reacted with nanotubes modified with oligonucleotides that are not complementary to the ssDNA bound to the gold electrodes. The degree of successes in connecting electrode pairs this way was considerably smaller than that achieved by using complementary sequences. The following Table compares the connection efficiency using complementary and non-complementary oligonucleotides.

| | Devices | Apparently | | Devices | % electrically |
|---|---|---|---|---|---|
| | examined | connected | Devices | electrically | connected |
| | by SEM | devices | measured | connected | devices |
| Complementary | 270 | 41 | 31 | 24 | 11.7 |
| Non-complementary | 288 | 7 | 7 | 2 | 0.7 |

FIG. 3A presents several current versus voltage curves measured for various devices obtained by the technique of the present invention using complementary oligonucleotides. The current magnitudes are different, yet share the common general features. Furthermore, the devices maintain their characteristic features over hundreds of measurements.

FIG. 3B shows current versus voltage curves measured for devices obtained using non-complementary oligonucleotides. The currents measured for these devices are smaller by more than an order of magnitude compared to the currents measured using complementary DNA.

It should be understood that the technique of the present invention leads to deposition of nanotubes and nanotube bundles between electrodes, a third of which being metallic or some enrichment process being involved [8,9]. Since the metallic fraction should dominate the transport characteristics, the conductivity obtained, at room temperature, stems from the DNA-mediated contacts. The importance of the chemical and geometrical nature of the metal/nanotube contact in determining the charge transport in carbon nanotube-based devices has already been emphasized [10-12].

It is important to note that with the technique of the present invention (metal-nanotube coupling via complementary DNAs), an electric current measured through the nanotube is larger by more than two orders of magnitude as compared to that observed for direct metal-nanotube contacts. Even assuming that in the case of the invented technique, a bundle instead of a single nanotube is used, since the bundle cannot contain more than tens of single tubes, the current measured still exceeds former measurements by more than an order of magnitude [13]. Hence, the importance of the nature of the nanotube-electrode contact is again emphasized. The technique of the present invention thus provides a method for producing nanotube-based electronic devices with high efficiency and proves that the transport properties of these devices are controlled by the chemical linker, namely the double stranded DNA.

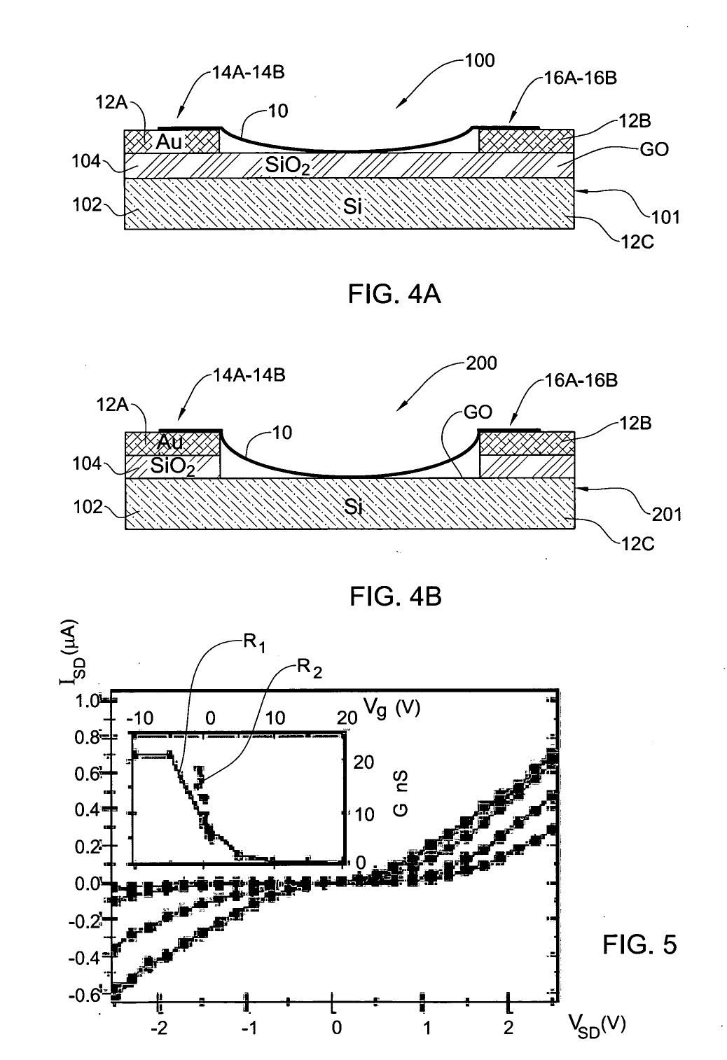

The inventors have utilized the above-described method (i.e., modifying nanotubes with single stranded DNA molecules and consequent hybridization with complementary DNA strands self-assembled onto metal contacts) to produce nanotube based field effect transistors with high yield. Reference is made to FIGS. 4A, 4B, 5, 6A, 6B and 7 showing experimental results of the transistors fabrication using this technique.

FIGS. 4A and 4B exemplify two transistor devices of the present invention. Each of these devices has a layer structure (silicon-based wafer) configured to define a gate oxide GO layer and a pair of electrodes 12A and 12B (source and drain electrodes); and the above-described device (1 in FIG. 1B) formed by these electrodes 12A and 12B connected by a nanotube or nanotubes bundle 10 via double stranded DNAs 14A-14B and 16A-16B. The transistor channel is defined by nanotube 10 located on top of gate oxide layer GO.

In device 100 of FIG. 4A, layer structure 101 includes a Si substrate layer 102 carrying a continuous 100-nm-thick SiO₂ dielectric layer 104, and a patterned metal (Au) layer defining two spaced-apart Au electrodes 12A and 12B on top of SiO₂ dielectric layer 104. Thus, here 100-nm-thick SiO₂ dielectric layer 102 presents gate dielectric GO.

In device 200 of FIG. 4B, layer structure 201 includes a Si substrate layer 102 carrying a SiO₂ dielectric layer 104 patterned to define spaced-apart SiO₂ dielectric regions 104A and 104B, and Au electrodes 12A and 12B on top of SiO₂ dielectric regions 104A and 104B, respectively. In this device, a native oxide of substrate layer 102 acts as a gate dielectric.

Devices 100 and 200 were fabricated by thermally growing 100 nm SiO₂ layer 104 on top of p-type boron-doped Si substrate 102 thus forming silicon-based wafer (101 and 201), followed by deposition of hundreds of metal pads (forming electrodes 12A and 12B). The metal pad is formed by depositing a 10 nm Ti adhesion layer, 30 nm Au, and 200 nm Ni protection layer. The metal pads were deposited by e-beam evaporation and standard lift-off process. A distance between the source and drain electrodes 12A and 12B (metal pads) is about 1.5-2 μm. In device 100, the entire layer 102 of highly doped silicon serves as a gate electrode 12C on insulator 104 (gate oxide GO). In device 200, inner part of layer 102 serves as a gate electrode 12C on insulator GO formed by native oxide within a surface region of layer 102 exposed to air.

Transistor devices 100 and 200 of two types were thus fabricated differing in the thickness of the oxide layer GO. Device 100, having a 100-nm-thick SiO₂ layer, was obtained by simply removing the Ni protection layer from the Au contacts by HCl wet etching. Device 200 was obtained by defining active areas between the source and drain electrodes by optical lithography, followed by selective dry etching of SiO₂ using a CHF₃-based RIE process. This process results in local openings in the thermally grown SiO₂ layer at positions intended to contain carbon nanotubes.

Thiolated oligonucleotides were self-assembled onto gold contacts 12A and allowed to hybridize with carbon nanotubes 10 modified with the complementary strands, as described above. The typical production efficiency of around 10% allowed for preparing hundreds of devices with relative ease.

SEM images of such devices reveal that in most cases individual carbon nanotubes and bundles of up to ten carbon nanotubes, bridge a gap between the source and the drain electrodes. The average diameter and length of the individual carbon nanotube used as judged by atomic force, transmission, and scanning electron microscopy are, respectively, 1.4 nm and 1.5 μm.

Electrical measurements were carried out using two Keithley Model 236 Source-Measure Units, which applied source-drain voltage, V_{sd}, and gate-drain voltage, V_{g}, and which measured source-drain current, I_{sd}, and leakage current, Iₗₑₐₖ, respectively. The latter was negligible for all the measurements.

FIG. 5 presents typical I_{sd}−V_{sd} curves, measured for different gate voltages, V_{g}, of the device having the 100 nm silicon oxide layer. Applying positive gate voltages corresponds to negatively charging the nanotube. As shown in the figure, applying negative gate voltage, V_{g}, leads to increase in the electric current through the nanotubes with the curves tending to become closer to linear, whereas applying positive gate voltage, V_{g}, leads to smaller currents and more S-like shaped curves. This behavior corresponds to p-type conduction in the nanotube, which is normally observed for carbon nanotubes based field effect transistors (CNTFETs), operating in ambient conditions.

The absolute values of the currents and, therefore, the differential conductance of different samples, vary significantly from device to device. This can be explained by the model of diffusive channel conduction [18] or by the Schottky contact barrier model [10, 19]. In the context of the diffusive channel model, differences in the number of semiconducting carbon nanotubes in the bundles, possible shunting of semiconducting carbon nanotubes by metal ones inside the bundles, and a different degree of carbon nanotube doping by oxygen or other environmental species, might affect the considerable variance in current characteristics. On the other hand, nanotube devices are known to be very sensitive to variation of the Schottky barriers on the carbon nanotube-metal interfaces due to adsorption of species on the metal pads [11, 20].

It should be understood that in the transistor devices of the present invention, both the nanotubes and the metal pads are covered by DNA molecules. Different extent of the organic coverage along the nanotubes or on the gold contacts may account for the observed current variations according to both models (diffusive channel conduction and Schottky contact barrier model).

The insert to FIG. 5 shows the variation of the differential conductance G=dI_{sd}/dV_{sd} at zero source-drain voltage (V_{sd}=0) as a function of the gate voltage, V_{g}, for the 100 nm-thick SiO₂ gate layer device of FIG. 4A (graph R₁) and the native oxide device of FIG. 4B (graph R₂).

In order to compare the carrier mobility in the transistors having different gate dielectric thickness, those few particular devices were selected from the large number of samples measured, which exhibit similar differential conductance at zero V_{g}.

Extracting the derivative dG/dV_{g} and calculating the gate capacitance allow for estimating such an important technological characteristic of Field Effect Transistors (FETs), as the field effect mobility, μ_{FE}, which determines the sensitivity of the device to variations of the gate voltage. This is a device-specific parameter, not a material-specific, and is usually used to compare the performance of different FETs.

Using the analytical formula for a cylinder over an infinite plane [21], and neglecting a factor of the order of unity due to presence of the dielectric only between the carbon nanotube and the gate electrode, but not on top of the nanotube, allows for estimating the capacitance per unit length of the carbon nanotube to be about 10 pF/m for the 100 nm-thick-gate-dielectric FETs (100 in FIG. 4A) and about 50 pF/m for the native-oxide FETs (200 in FIG. 4B). The corresponding mobilities are, therefore, μ_{FE}˜25 cm²V⁻¹s⁻¹ and μ_{FE}˜35 cm²V⁻¹s⁻¹, for thicker and thinner gate dielectric, respectively.

The field-effect mobility of the devices of the present invention depend only slightly on the dielectric thickness and is also slightly lower than values reported for back-gate CNTFET structures with SiO₂ gate dielectric [18]. The inventors have attributed both phenomena to the geometry of the devices, where an interface between the carbon nanotube and Au-electrode is located on the top or on the edge of the metal contact pad. This leads to partial screening of the contact Schottky barriers (which determine the conduction in the devices) from the gate voltage.

The above is further confirmed by the results obtained from measuring the inverse subthreshold slope S=(d(log I_{sd})dV_{g})⁻¹. For the channel-determined conduction, as in the case of conventional long-channel MOSFETs, the inverse subthreshold slope S depends linearly on the temperature but is independent of the source-drain voltage [22]. The temperature dependence of the inverse subthreshold slope of CNTFETs is reported to be very weak and is far from being linear, confirming Schottky-barrier nature of the CNT conduction [10, 12].

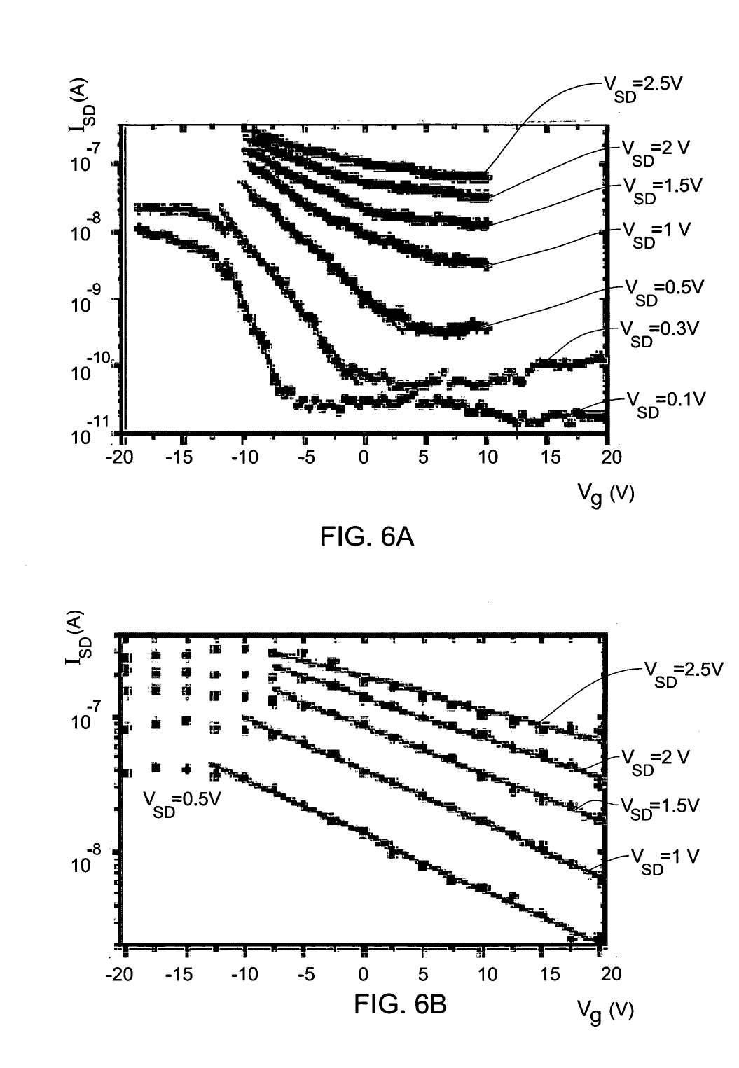

FIGS. 6A and 6B illustrate the dependence of the source-drain current, I_{sd}, on the gate voltage at subthreshold region, measured at different source-drain voltages, V_{sd}, for respectively the 100 nm gate dielectric layer device (100 in FIG. 4A) and the native oxide layer device (200 in FIG. 4B). Linear regions of the curves are used to extract the subthreshold slope. The “on-state” and “off-state” currents Iₒₙ and I_{off}, as well as the slope at the linear region of the curves, clearly depend on the source-drain voltage, V_{sd}. The ratio Iₒₙ/I_{off} changes by at least an order of magnitude with V_{sd} in the region 0.1-2.5V, and reaches 10³ at V_{sd}=100 mV. The inverse subthreshold slope was calculated from the linear regions of the log I_{sd} vs V_{g} curves, and its absolute values are of the same order of magnitude and only slightly higher compared to similar back-gate structures. The somewhat higher value could be explained by the above-described effect of partial screening by the contact pads.

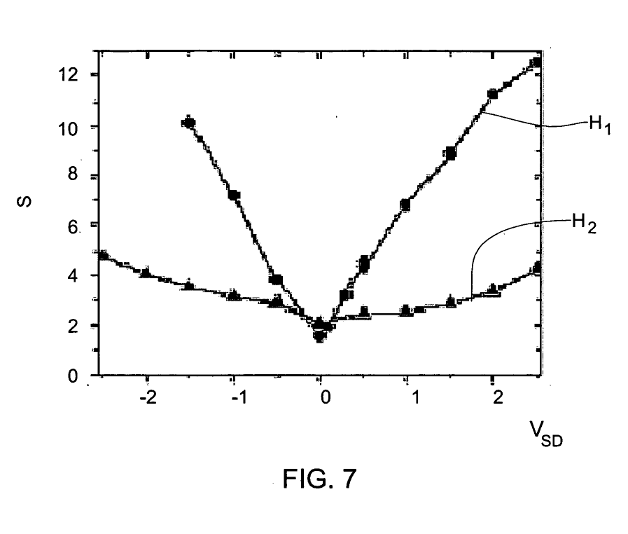

FIG. 7 illustrates an inverse subthreshold slope, S, as a function of source-drain voltage, V_{sd}. In the figure, graph H₁ corresponds to the 100 nm-thick SiO₂ gate layer device (100 in FIG. 4A), and graph H₂ corresponds to the native oxide device (200 in FIG. 4B). This variation of inverse subthreshold slope, S, with the source-drain voltage, V_{sd}, provides yet another indication that the conductance in CNTFETs is indeed determined by the interface Schottky barriers.

Thus, the present invention provides for producing CNTFETs with high yields of about 10% by methods of self-assembly using the natural process of DNA hybridization. Electronic devices fabricated using this method behave like those made using direct metal-carbon nanotube contacts. The inverse subthreshold slope of the CNTFETs depends on the source-drain voltage applied to the device, confirming that the conductance of CNTFETs is determined by the Schottky barriers at the interfaces between the carbon nanotubes and the electrodes.

The technique of the present invention provides for self-assembling different DNAs on different electrodes even at very small distances between the electrodes (distances below 1 μm), thus enabling to produce a pattern with very small features. An example of this technique is illustrated in FIGS. 8A-8B.

FIG. 8A shows an array of spaced-apart electrodes, three such electrodes E₁, E₂ and E₃ in the present example, on top of a dielectric substrate 40. The electrodes are separately addressed with a voltage supply. First, all the electrodes, except for electrode E₁, are kept at a certain negative voltage (e.g. −1.8V). When a drop 42 containing thiolated ssDNA of a given sequence (DNA-1) is deposited on the electrodes array, the thiolated ssDNA reacts solely with electrode E₁. The electrodes are then washed to remove all unbound DNA-1. Then (FIG. 8B), the voltage supply is modified such that a certain negative voltage is applied to electrode E₃, and a drop 44 containing a second thiolated ssDNA of a given sequence (DNA-2) is deposited on the electrodes array. Since electrode E₁ is already DNA bound and electrode E₃ is under the negative voltage supply, this DNA-2 binds only to electrode E₂. In such a way, selective coupling of different ssDNA sequences to different electrodes may be achieved. It should be understood that this technique may be applied such that more than one electrode may be coupled to one ssDNA sequence, while one or more of the other electrodes would be bound to a different sequence, and that this may be modified to apply to any number of electrodes or sequences.

The technique of the present invention also provides for self-assembling different DNAs on a single nanotube, which would be useful for binding to electrodes bearing different ssDNA sequences. Examples of this technique are illustrated in FIGS. 9A-9C.

FIG. 9A shows nanotubes 10 bound to a substrate 50 through DNA connection, such that one end 10A of each nanotube is bound to the substrate 50 and the second end 10B remains unbound. The nanotubes are immersed in a solution containing a first ssDNA sequence (DNA-3). As described above, a reaction takes place wherein DNA-3 is bound to the free end 10B of the nanotubes. Then nanotubes 10 are separated from substrate 50 such that end 10A is freed and all unbound DNA-3 is removed. The separation occurs by washing the sample with pure water causing the dehybridization of the DNA double strands.

The nanotubes 10 are then immersed in a solution containing a second ssDNA sequence (DNA-4) and allowed to react with the DNA, resulting in binding of DNA-4 to the freed end 10A of nanotube 10 (FIG. 9B).

It should be understood that in case the DNA solution includes more than one DNA sequence, then more than one DNA sequence may be bound to each end of the nanotube, as shown in a self explanatory manner in FIG. 9C. In such case, the step of binding and removing the nanotubes 10 from the substrate 50 is omitted.

Fabrication of devices from individual carbon nanotubes (SWNTs) is related to the uncontrolled chirality and length of the SWNTs, and their tendency to bundle. Networks of SWNTs positioned between electrode pairs can occupy more space, yet can be easier to obtain. Also, inventors have found that the electrical properties of devices obtained using the self-assembly method can be quite uniform despite the structural non-uniformity of such devices.

In some preferred embodiments of the SWNT-utilizing electronic devices, the electronic properties of the SWNTs are uniform. Also, in some preferred embodiments of the SWNT-utilizing electronic devices, the devices are made with multiple SWNT contacts. In such cases, the electronic properties are averaged and a uniform performance of the devices is achieved, despite various non-uniformities amongst different SWNTs forming the devices, and the variability in the SWNT contacts.

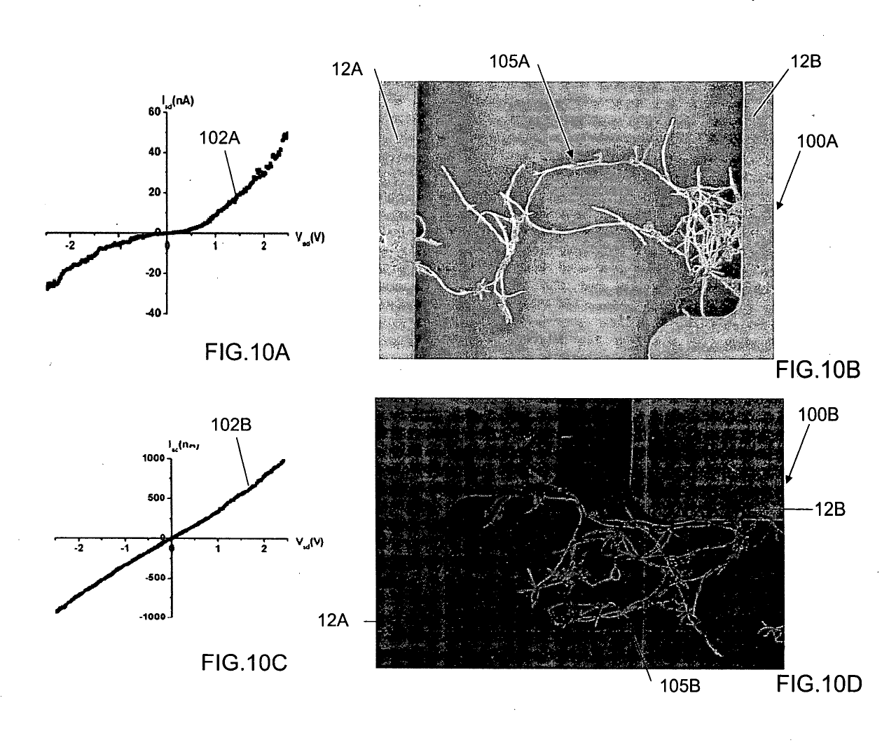

In reference to FIGS. 10A-10D there are shown images 10B and 10D of two electronic devices 100A and 10B, respectively, and I-V curves 102A and 102B (FIGS. 10B and 10D) for these devices. Devices 100A and 100B are based on SWNT networks 105A and 105B, respectively. In each of the devices the corresponding SWNT network connects electrodes 12A and 12B. I-V curve 102A of device 100A is highly non linear and not symmetric in the range of −3V to +3V bias voltage. I-V curve 102B of device 100B features a linear I-V dependence with a current exceeding by an order of magnitude the current achieved for network 105A. In this connection, it should be noted that network 105B involves a very large number of SWNTs. When a contact between electrodes is formed by a SWNT network, more than one conduction path can be established. Such conduction paths may include junctions between various types of SWNTs. Multiple parallel conduction channels reduce the total resistance, especially if metallic pathways are formed, however junctions formed between metallic and semi-conducting SWNTs may increase the resistance due to formation of Schottky barriers. In the case of conducting channels that involve metallic SWNTs an ohmic-like I-V curve is observed, similar to that demonstrated in FIG. 10C.

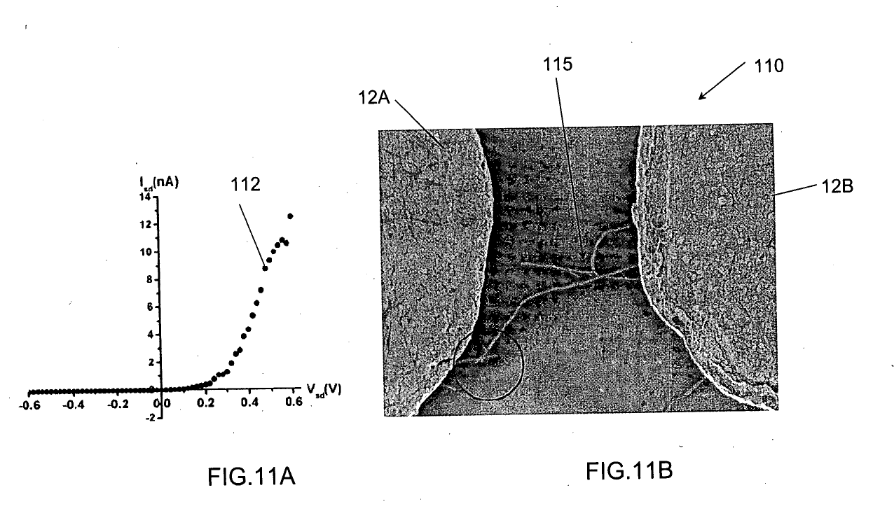

FIGS. 11A and 11B show an image of an electronic device 110, having a SWNT network 115 and two electrodes 12A and 12B (FIG. 11B), and an I-V curve 112 of this device (FIG. 11A). SWNT network 115 of device 110 involves a single junction 118 between SWNTs. I-V curve 112 of device 110 demonstrates a diode-like behavior. The I-V curve and the small magnitude of the current can be explained by the formation of a Schottky barrier at the contact point between a metallic SWNT and a semiconducting SWNT. The formation of a junction between SWNTs causes an increase of the resistivity and increases the contribution of leakage through the substrate to the measured current. This explains the current which is an order of magnitude smaller then the current shown in FIG. 2C.

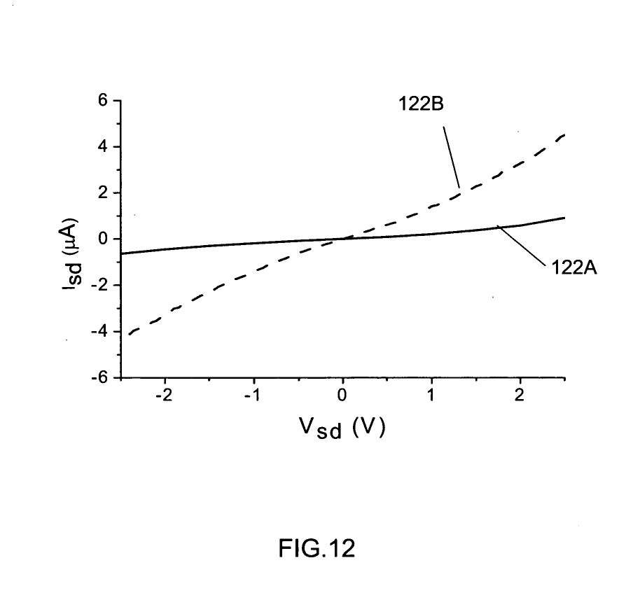

FIG. 12 demonstrates two I-V curves, 122A and 122B, obtained for a device of the type described in reference to FIGS. 10A and 10B (i.e. for a device which network contains multiple conducting channels for connecting two electrodes). Curve 122A was obtained before, and curve 122B was obtained after a mild thermal treatment (e.g. 200° C. for 30 min.) applied to the device. As it can be seen from comparing the two curves, even a mild thermal treatment can result in an up to two orders of magnitude increase in the current at a given bias voltage. The increase in the current is due to the destruction of the DNA molecules binding the SWNT network and the electrodes, and the formation of a direct gold-SWNT contact. In FIG. 12 the shape of the I-V curve remained the same even though the current had increased. Hence, the DNA molecules act as resistors, but do not change significantly the density of states of the SWNT and therefore do not affect the shape of the barrier at the metal-SWNT contact.

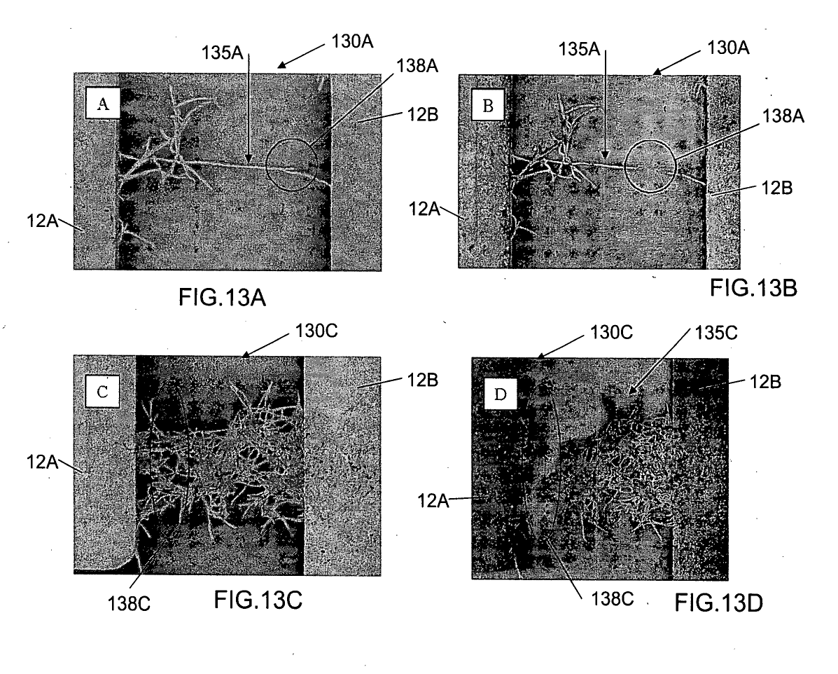

FIGS. 13A-13D present four scanning electron microscopy (SEM) images of two devices 130A and 130C of the kind specified above. Electrodes are denoted 12A and 12B in all four figures. In FIG. 13A and FIG. 13C devices 130A and 130C, having SWNT networks 135A and 135C, respectively, conduct electrical current. FIGS. 13B and 13D illustrate an effect of high currents on devices 130A and 130C: the effect can be seen as damaged regions 138A and 138C, respectively. The devices can function for very long time as long as the current through them does not exceed the break-down current. Inventors have found that the current induced damage occurs mostly along the SWNTs' lengths and especially at their junctions (i.e. at SWNT/SWNT interfaces) rather than at SWNT/electrode interfaces. Current-induced breakdown of the devices therefore depends not only on the number of SWNTs connecting the electrodes but on the manner in which the SWNTs bridge the gap, namely direct contact or through SWNT junctions. Despite the fact that the connection between the SWNT and the electrodes was done with a short double stranded DNA (typically 26 base-pair about 8.8 nm long), the DNA was not the limiting current component and devices were not burned at the SWNT-electrode contact. Since many DNA single strands are bound at the end of each SWNT, it can be estimated that up to ten DNA strands are involved in each contact made between the SWNT and the electrodes. Despite this parallel contact, the current density through each of the DNA contact is very high.

In the case of single SWNT connector (FIGS. 13A and 13B), the current induced break-down occurred at a current of about of few hundreds of nA. When the devices were treated thermally (as described above) the break-down occurred at current of up to 20 μA. This is due to the following two effects: decomposition of the DNA at the contact, resulting in a better metal-SWNT contact, and annealing of defects on the SWNT. Such an annealing causes SWNT to be more uniform. FIGS. 13C and 13D show a case where a network of multiple SWNTs connects the electrodes, a situation obviously involving multiple junctions, a few of which are expected to be highly resistive. Nevertheless, the current induced break-down region 138C is highly localized and spatially distinct. The aforementioned higher resistance of SWNT/SWNT junctions also explains that the current driven breakdown is observed at such highly localized regions. Previous work on current saturation and electrical breakdown in multiwall carbon nanotubes showed that current induced breakdown relates to substantial carbon loss [47]. In most cases the breakdown process involves excitation of high energy optical or zone boundary phonons leading to self heating and reaction with oxygen. The highly localized breakdown involving multiple tubes, shown in FIG. 13D, supports the role of local heating in breakdown.

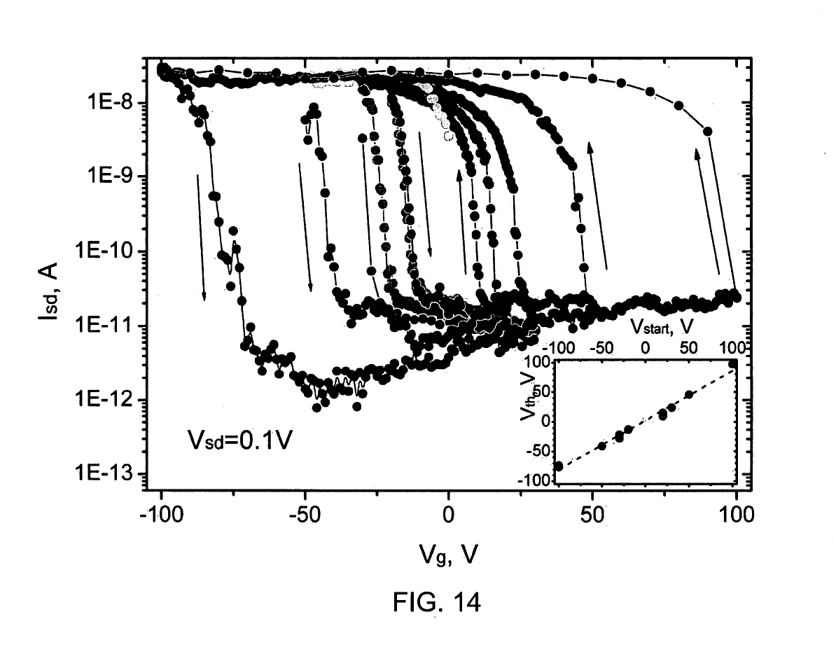

For further probing of the electronic properties of the self-assembled devices, field effect transistors (FET) with back-gates were fabricated and investigated. The hysteretic behavior of the I-V characteristics of the carbon nanotube FETs (CNTFETs) that has been reported [42] before is manifested in the shift of the threshold voltage. This shift is attributed to a redistribution of charges in the vicinity of the nanotube caused by the applied gate voltage. In FIG. 14 hysteresis curves measured by the inventors for sweeps of the gate voltage in different ranges are presented. The anticlockwise direction of I_{sd} vs V_{g} hysteresis loops indicates that the injection of the charges into traps in and on the surface of the gate dielectric is the cause of the hysteresis. The threshold voltage clearly depends on the starting voltage of each sweep and is very close to the latter up to high values of about 100V as shown in the insert to FIG. 14. The hysteresis observed in the measurements performed by the inventors is thus much wider than those observed by other groups [43, 44]. It should be noted here that in devices fabricated by the inventors DNA molecules are chemisorbed at defect sites along the SWNTs. The adsorbed molecules can serve as additional surface charge traps, which increase the total density of traps and, consequently, lead to a wider hysteresis loop.

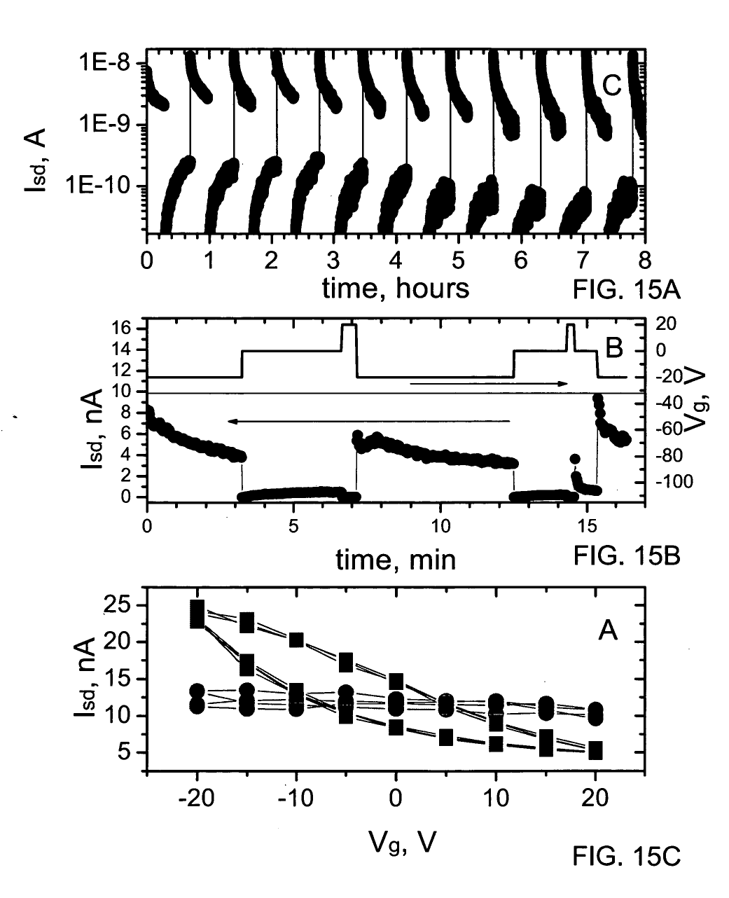

Stability of the device current under constant voltage is desirable for any practical application of the observed hysteresis in a memory device. FIG. 15A presents the hysteresis loops measured with different delays between the voltage setting and the current measurement. It is clearly seen that for long enough delays the hysteresis almost disappears, i.e. the current relaxes to its equilibrium value after the charges are released from their traps. The similar effect is observed when the delays are made between the individual V_{g} points with no delay between the voltage setting and the current measurement (not shown).

FIG. 15B shows several write-read cycles of the invented CNTFETs while switching the gate voltage between +20 V, 0 V and −20 V, whereas FIG. 15C presents the variation of the device current on long-range scale while switching the gate voltage between +20 V and −20 V. These graphs confirm that the characteristic timescale of the traps discharge is in the range of hours, thus confining the action of the memory devices to this timescale. It should be noted however that the duration of the write pulse is preferably long enough in order to produce measurable effects. After 1-sec-pulse of the gate voltage to ±20 V the I_{sd} remained the same as before the pulse (not shown).

Hence, in the invented devices, the effect of hystheresis is pronounced and the electric potential range in which hystheresis is observed is broader than reported before. The hystheresis effect is attributed to charging of defect states in the vicinity of the nanotubes. The lifetime of the charge on the defects is shorter. These findings are explained by assuming that the adsorbed DNA blocks the deep traps, yet increases the total number of traps. These effects make possible for the chemical modification and control of the hystheresis properties.

In order to compare the carrier mobility in CNTFETs having different values of the gate dielectric thickness, a few particular devices have been picked out of the large number of samples exhibiting similar differential conductance, G=dI_{sd}/dV_{sd}, at zero V_{g}. Extracting the derivative dG/dV_{g} and calculating the gate capacitance allowed to estimate an important technological characteristic of FETs, the field effect mobility, μ_{FE}, which determines the sensitivity of the device to variations of the gate voltage. This is a device-specific parameter, not a material-specific, and is usually used to compare the performance of different FETs [42]. Using the analytical formula for a cylinder over an infinite plane [21], and neglecting a factor of the order of unity due to presence of the dielectric only between the CNT and the gate electrode but not on top of the nanotube [46], allowed to estimate the capacitance per unit length of the CNT as being about 10 pF/m for the 100-nm-thick-gate-dielectric FETs and about 50 pF/m for the native-oxide FETs. The corresponding mobilities are, therefore, μ_{FE}˜25 cm²V⁻¹s⁻¹ and μ_{FE}˜35 cm²V⁻¹s⁻¹, for thicker and thinner gate dielectric, respectively. The field-effect mobility of the devices depends only slightly on the dielectric thickness and is also slightly lower than values reported for back-gate CNTFET structures with SiO₂ gate dielectric [17]. Both phenomena can be attributed to the geometry of the devices, where CNT/Au interfaces are located on the top or on the edge of the metal contact pads. This leads to the partial screening of the contact Schottky barriers (which determine the conduction in the CNTFETs) from the gate voltage.

This conclusion is confirmed by the results obtained from measuring the inverse subthreshold slope S=(d(log I_{sd})/dV_{g})⁻¹, which is already shown in FIG. 7. For channel-determined conduction, as in the case of conventional long-channel MOSFETs, the inverse subthreshold slope S depends linearly on the temperature but is independent of the source-drain voltage [22]. The temperature dependence of the inverse subthreshold slope of CNTFETs is very weak and is far from being linear, confirming Schottky-barrier nature of the CNT conduction [10, 12]. It provides yet another indication that the conductance in CNTFETs is determined indeed by the interface Schottky barriers.

Thus, the present invention provides a simple and effective technique of producing an electronic device or an array of such devices, by coupling an electrically conductive element to an elongated carbon or inorganic semiconductor based nanostructure (e.g. at least one single-walled carbon nanotube (SWNT) or SWNTs bundle, or a network of SWNTs), by a biological binder (e.g. DNA serving as a chemical linker, using a natural process of the DNA hybridization). Also, the present invention provides for using this technique to fabricate a transistor or an array of transistors.

Those skilled in the art will readily appreciate that various modifications and changes can be applied to the embodiments of the invention as hereinbefore described without departing from its scope defined in and by the appended claims and their equivalents. Also, it is to be understood that the phraseology and terminology employed herein are for the purpose of description and should not be regarded as limiting.

1. An electronic device comprising
an elongated carbon or inorganic semiconductor based nanostructure
selectively coupled to a first electrode, via a biological binder formed by first and second moieties attached to the nanostructure and the electrode respectively and being capable of specifically interacting with one another, at least one of said first and second moieties being a biological moiety.

2. (canceled)

3. The device of claim 1, wherein the first and second moieties are recognizable by one another thereby enabling the selective coupling providing stable biological binding.

4. The device of claim 2, wherein the interaction between said first moiety with the recognizable second moiety is direct.

5. The device of claim 1, wherein the other than the biological moiety of said first and second moieties is a biological moiety or an inorganic substance.

6. The device of claim 1, wherein said at least one biological moiety comprises at least one of the following: a peptide based molecule, a nucleic acid based moiety, or a modification of the peptide based molecule or nucleic based moiety.

7. The device of claim 5, wherein the peptide based molecule comprises at least one of the following: peptide, polypeptide, protein, and glycoprotein.

8. The device of claim 5, wherein the nucleic acid based moiety comprises at least one of oligonucleotide and polynucleotide.

9. The device of claim 1, wherein the specific interaction between said first and second moieties comprises at least one of the following interactions: protein/protein, nucleic acid/protein and nucleic acid/nucleic acid interaction.

10. The device of claim 1, wherein the first and second moieties are biological moieties comprising at least one of the following combinations: enzyme/substrate, antigen/antibody, ligand/receptor, nucleic acid sequence/nucleic acid sequence, nucleic acid sequence/nucleic acid biding proteins, sugar/lectin, enzyme/inhibitor, enzyme/co-factor.

11. The device of claim 9, wherein the nucleic acid sequence in the biological moieties comprises one of the following: a sequence composed of DNA nucleotides, a sequence composed of RNA nucleotides, a sequence composed of DNA and RNA nucleotides, natural nucleotides, chemically modified nucleotides, and synthetic nucleotides.

11. The device of claim 1, wherein the biological binder is in the form of a double strand.

12. The device of claim 1, wherein said biological binder is formed as a result of natural hybridization of free ends of two complementary single stranded nucleic acids sequences, opposite ends of which being adsorbed to, respectively, the electrically conductive element and the elongated nanostructure.

13. The device of claim 1, comprising a second electrode coupled to an opposite end of the elongated nanostructure by a second biological binder formed by two binding moieties which are attached to said opposite end and the second electrode respectively and which are of a kind capable of specifically interacting with one another, at least one of said two moieties being a biological moiety.

15. The device of claim 14, wherein the second biological binder is formed as a result of natural hybridization of free ends of two complementary single stranded nucleic acids sequences, opposite ends of which being adsorbed to, respectively, the first and second electrodes and the respective ends of the elongated nanostructure.

16. The device of claim 14, configured as a transistor device comprising a layer structure defining said first and second electrodes serving as source and drain electrodes of the transistor, a gate electrode on a dielectric, and a transistor channel formed by said elongated nanostructure located on said dielectric and interconnected between the source and drain electrodes via the first and second biological binders.

17. The device of claim 16, wherein said layer structure comprises a semiconductor substrate carrying a continuous layer of said dielectric, and the source and drain electrodes arranged in the spaced-apart relationship on top of said dielectric layer, the semiconductor substrate serving as the gate electrode on the dielectric.

18. The device of claim 16, wherein said layer structure comprises a semiconductor substrate carrying two spaced-apart regions of said dielectric spaced by a region of said substrate, and the source and drain electrodes on top of said dielectric regions, respectively, a native oxide of the surface region of said substrate within the space between the dielectric regions serving as the dielectric for the gate electrode defined by the inner material of the semiconductor substrate.

19. An electronic device comprising
at least first and second spaced-apart electrodes connected to each other via a carbon or inorganic semiconductor based elongated nanostructure,
wherein each end of the nanostructure carries a moiety adsorbed thereto which is capable of specifically interacting with another moiety adsorbed to the respective electrode,
at least one of the moieties being a biological moiety,
the two coupled moieties thereby presenting a biological binder chemical linking the nanostructure to the electrode.

20. A field effect transistor device comprising
spaced apart source and drain electrodes, a gate electrode on a dielectric, and an elongated nanostructure which is interconnected between the source and drain electrodes and thereby forms a transistor channel,
opposite ends of said nanostructure being connected to the source and drain electrodes, respectively, via two biological binders,
the biological binder being formed by a first moiety adsorbed onto the respective electrode and a second moiety adsorbed onto the respective end of the nanostructure,
at least one of the first and second moieties being a biological moiety,
the first and second moieties being recognizable by one another.

21-25. (canceled)
